(19) United States
(12) Patent Application Publication
(10) Pub. No.: US 2019/0090670 A1
(43) Pub. Date: Mar. 28, 2019

(54) Hinged Forceps with Finger Grips

(71) Applicant: Join Y. Luh, Arcata, CA (US)

(72) Inventor: Join Y. Luh, Arcata, CA (US)

(21) Appl. No.: 16/204,122

(22) Filed: Nov. 29, 2018

Related U.S. Application Data
(63) Continuation-in-part of application No. 14/881,895, filed on Oct. 13, 2015.
Application No. 14/881,895 is a continuation of application No. 12/226,301, filed on Oct. 23, 2009, now abandoned.
(60) Provisional application No. 60/790,916, filed on Apr. 11, 2006.

(51) Int. Cl. A47G 21/10; A45D 26/00; A61B 17/30; A47J 43/28; A61B 17/00

(52) U.S. Cl. CPC A47G 21/10; A45D 26/0066; A61B 2017/00438; A47J 43/283; A61B 17/30

(57) ABSTRACT

Hinged forceps which aid in grasping and moving various objects such as food items or tools. The hinged forceps generally include a plurality of arms which are interconnected by a hinge such that each of the arms may independently pivot with respect to the hinge. Each of the arms may include one or more finger grips which may be positioned at various locations along the length of each arm; the finger grips being adapted to support one or more fingers of the user. The hinge interconnecting the arms may allow uninhibited movement of one arm relative to the other arm. Each arm may be adjustable to spread or contract with respect to the other arms such that the effective distance between arms may be adjusted. The finger grips may be adjustably mounted on an arm such that they are capable of rotating in position and/or being repositioned on the arm.

### CROSS REFERENCE TO RELATED APPLICATIONS

This application is a continuation-in-part of U.S. patent application, Ser. No. 14/881,895, entitled “Hinged Device for Transferring Food” filed on Oct. 13, 2015, which is a continuation of Ser. No. 12/226,301, entitled “Hinged Forceps”, filed Oct. 23, 2009, which is a 371 of PCT Application PCT/US2007/066427, filed Apr. 11, 2007, which claims priority to U.S. Provisional Application Ser. No. 60/790,916, filed Apr. 11, 2006.

### STATEMENT REGARDING FEDERALLY SPONSORED RESEARCH OR DEVELOPMENT

Not applicable to this application.

### BACKGROUND

### Field

The present invention relates to hinged forceps. More particularly, the invention relates to hinged forceps that include finger grips.

### Related Art

Any discussion of the related art throughout the specification should in no way be considered as an admission that such related art is widely known or forms part of common general knowledge in the field.

Forceps are commonly used in medical procedures, engineering applications, food-service applications, and beauty applications. Forceps usually include two arms fused together at an end or joined with a spring mechanism. The fused joint or spring mechanism causes inhibited motion of the arms of the forceps. In such instances, movement of the arms with respect to each other is resisted until a force is applied to the forceps to overcome the resistive force of the spring mechanism or fused joint. Thus, a user must constantly apply pressure to the arms to retain the altered position of the arms with respect to each other. In addition, a user must maintain pressure on the forceps to control the forceps while applying pressure to the forceps to retain an object. With repetitive use, this need to apply pressure to the arms will cause fatigue. A user often must also worry about dropping the forceps during use. For example, during long surgeries, doctors may experience hand strain that may cause loss of control of the forceps. This even occurs in the so-called “reverse” forceps where applying pressure opens the forceps. In the beauty industry, cosmetologists who use forceps to remove hair also may experience hand fatigue since they have to repetitively apply pressure to the arms of the forceps during use. In the food industry, tongs used during cooking or in buffet lines may result in hand fatigue from repetitive application of pressure by the hand being positioned too close to the tongs when picking up food using the tongs. Customers with arthritis often struggle with the tongs provided in restaurants (e.g., such as at serving buffets).

### SUMMARY

An example embodiment is directed to a Hinged Forceps with Finger Grips. The Hinged Forceps include at least two arms, one or more finger grips, and a hinge coupling the two arms to each other. At least one finger grip may be positioned on at least one of the arms. The finger grips may be attached to the arms using a swivel. The hinge may be positioned at an end of each of the arms. The hinge may restrict the range of motion of the arms with respect to each other. The hinge may function to allow a predetermined separation between the arms of the device. In some embodiments, the hinge may allow at least 30 degrees, 45 degrees, 60 degrees, at least 90 degrees or at least 180 degrees of separation between the arms. In certain embodiments, the hinge may allow substantially uninhibited movement of one of the arms with respect to the other arms. One or more of the arms may including protrusions. In an embodiment, the forceps may be formed of a material capable of being sterilized. In some embodiments, the forceps may be formed of a disposable material. In an embodiment comprising at least three arms, the arms may move independently of each other. In other embodiments, one or more arms may move in unison.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIG. 1 depicts an embodiment of a pair of forceps.

FIG. 2A depicts an embodiment of a pair of forceps in a partially open position.

FIG. 2B depicts an embodiment of a pair of forceps opened such that the arms are separated by 180 degrees.

FIG. 3 depicts an embodiment of forceps with three finger grips.

FIG. 4A depicts an embodiment of a pair of forceps with flexible finger grips.

FIG. 4B depicts an embodiment of forceps with circular finger grips.

FIG. 5 depicts an embodiment of a pair of forceps for use as an eating utensil.

FIG. 6A depicts an embodiment of forceps comprising three arms.

FIG. 6B depicts an embodiment of forceps comprising four arms.

FIG. 6c depicts an embodiment of forceps comprising four arms including an adjustment mechanism allowing the arms to spread.

FIG. 7 depicts a top perspective view of an alternate embodiment wherein the finger grips are attached in a manner that allows them to swivel.

FIG. 8 depicts a bottom perspective view of an alternate embodiment wherein the finger grips are attached in a manner that allows them to swivel.

FIG. 9 depicts a side view of an alternate embodiment wherein the finger grips are attached in a manner that allows them to swivel.

FIG. 10 depicts a top view of an alternate embodiment wherein the finger grips are attached in a manner that allows them to swivel.

FIG. 11 depicts a bottom view of an alternate embodiment wherein the finger grips are attached in a manner that allows them to swivel.

FIG. 12 depicts a front view of an alternate embodiment wherein the finger grips are attached in a manner that allows them to swivel.

FIG. 13 depicts a rear view of an alternate embodiment wherein the finger grips are attached in a manner that allows them to swivel.

FIG. 14 depicts a side view of an alternate embodiment wherein the finger grips are attached in a manner that allows them to swivel wherein the arms are separated.

FIG. 15 depicts a side view of an alternate embodiment wherein the finger grips are attached in a manner that allows them to swivel wherein the arms are have greater separation than FIG. 14.

FIG. 16 depicts the interior face of the upper arm of an alternate embodiment wherein the finger grips are attached in a manner that allows them to swivel.

FIG. 17 depicts the interior fact of the lower arm of an alternate embodiment wherein the finger grips are attached in a manner that allows them to swivel.

FIGS. 18A, 18B, and 18C depict an embodiment wherein the finger grips are configured to be repositioned along a slot in the upper arms at three different locations.

FIGS. 19A, 19B, and 19C depict an embodiment wherein the finger grips are configured to be repositioned along a slot and rotate showing the finger grip at three different orientations.

FIG. 20 is a perspective view of an embodiment comprising teeth on the ends and sides of the distal end of each arm.

FIG. 21 is a side view of an embodiment comprising teeth on the ends and sides of the distal end of each arm with the arms separated.

FIG. 22 is a top view of an embodiment comprising teeth on the ends and sides of the distal end of each arm with the arms separated.

FIG. 23 depicts an embodiment wherein the distal ends (ends furthest from the hinge) of the forceps are shaped like eating utensils.

FIG. 24A illustrates an embodiment being grasped by a user.

FIG. 24B depicts an embodiment being moved toward an object to be picked up.

FIG. 24C depicts an embodiment grasping onto an object.

FIG. 24D depicts an embodiment being lifted after grasping onto an object.

### DETAILED DESCRIPTION

### A. Overview

An example Hinged Forceps with Finger Grips generally comprises: a plurality of elongated arms coupled to a hinge with each arm comprising at least one finger grip attached to the exterior of its arm. Each finger grip may be attached to its respective arm in a manner that allows it to swivel and/or be repositioned along the length of its arm. The distal end of each arm (end furthest from the hinge) may includes one or more gripping protrusions and/or be shaped like eating utensils.

### B. Hinged Forceps

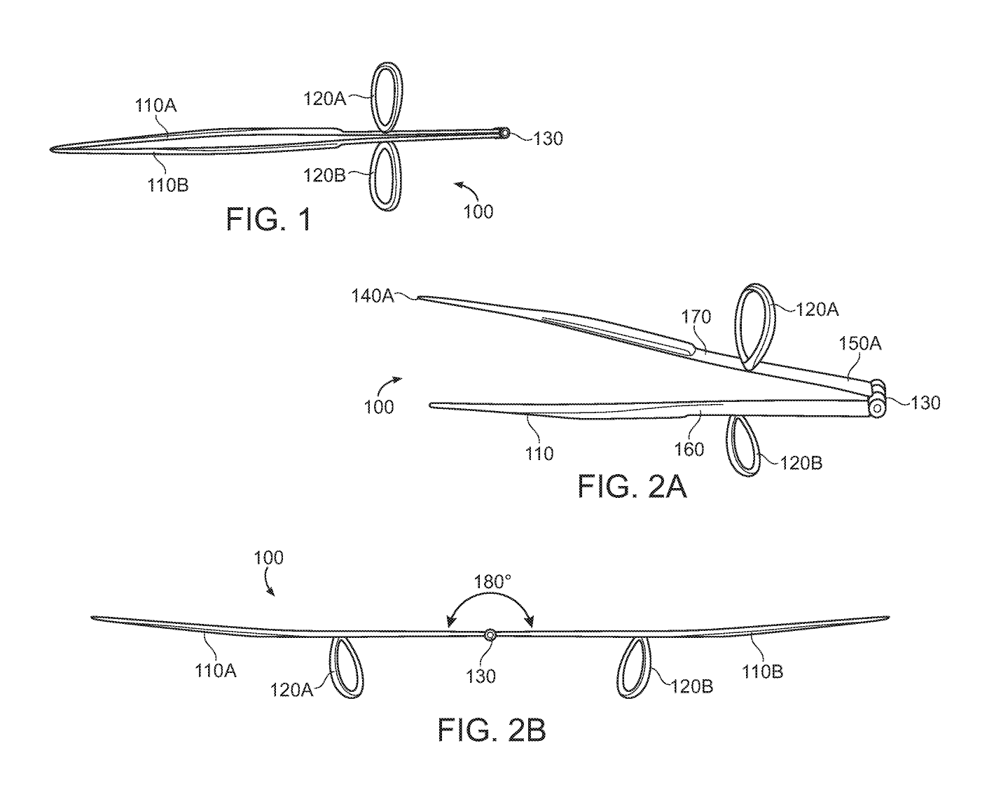

In various embodiments, exemplary forceps 100 may include arms 110, one or more finger grips 120, and a hinge 130, as depicted in FIG. 1. Forceps may be used to hold objects, close areas, and/or open areas. For example, during surgery, forceps 100 may be used to hold a blood vessel, an organ, or tissue; clamp an area, an organ, or a blood vessel closed; or open an area for surgery. Forceps 100, such as tweezers, may be used in the beauty industry to remove hair. Forceps 100, such as tongs, may be used in the food service industry to allow a user to hold and transfer food. Forceps 100, such as chopsticks, may be used during cooking and/or dining.

Forceps 100 may be formed from a variety of metal and/or non-metal materials. In an embodiment, forceps 100 may be formed of non-metal materials such as plastic, wood, rubber, ivory, or combinations thereof. Forceps 100 may be formed such that they are disposable. Forceps 100 may be formed from one or more materials that may be sterilized. Forceps 100 may be formed from stainless steel or another autoclaveable material. Forceps 100 may be formed of a food-safe material.

In some embodiments, forceps 100 may include two or more arms 110 (e.g., 110A, 110B, 110C). In some embodiments, forceps 100 may include three or more arms 110A, 110B, 110C, 110D. An arm 110 may be an elongated member or a conduit. An arm 110 may have a substantially square, rectangular, circular, oval, oblong, or irregular cross-sectional area. The length of an arm 110 may be selected based on the application of the forceps 100. For example, arms 110 in forceps 100 for clamping a region of the body may be larger than arms 110 in forceps 100 for closing a capillary. In an exemplary embodiment, at least a portion of an arm 110 may taper. For example, an arm 110 may taper away from hinge 130 and decrease in width towards a distal end 140. In another exemplary embodiment, an arm 110 may taper towards a proximate end 150 near hinge 130.

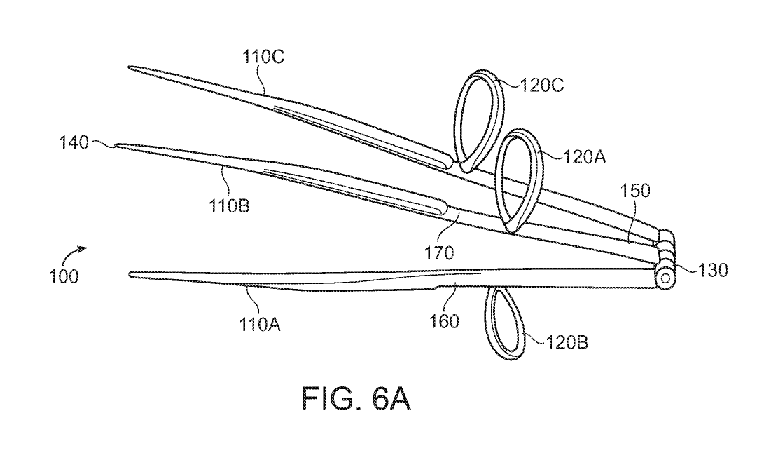

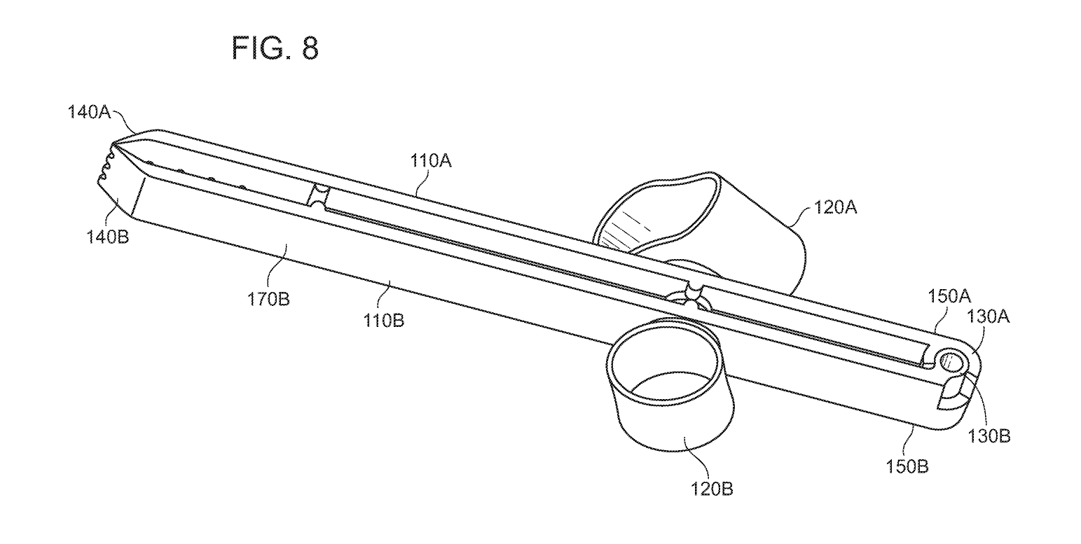

An arm 110 may have a distal end 140 that is furthest from hinge 130 and a proximate end 150 that is coupled to another arm via a hinge 130, as depicted in FIGS. 1 to 12. A distal end 140 of an arm 110 may have a shape designed to facilitate gripping. For example, a distal end 140 of an arm 110 may be serrated comprising a plurality of teeth 190. The area near distal end 140 of an arm 110 may be texturized to facilitate gripping. In an embodiment, an interior surface 160 of the arm 110 may be texturized. The area near distal end 140 of an arm 110, including an interior surface 160 of the arm 110 may include protrusions, ribs, a cross-hatch pattern, or other texture to facilitate gripping.

In some embodiments, a portion of a distal end 140 of an arm 110 may be formed of and/or coated with a material configured to facilitate gripping. For example, forceps 100 may include a rubber tip at the distal end 140 of each arm 110. In certain embodiments, at least a portion of a distal end 140 of an arm 110 may be formed of and/or coated with a material configured to inhibit damage to objects 210 being grasped.

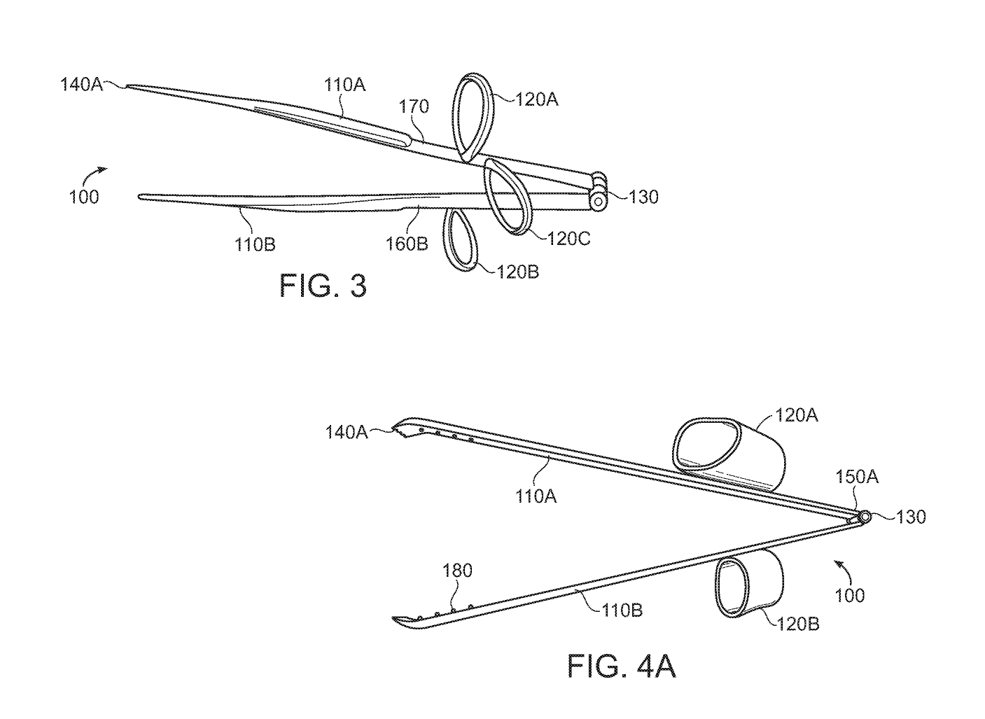

At least one finger grip 120 may be positioned on each arm 110. In an embodiment, two or more finger grips 120A, 120C may be positioned on an arm 110A, as depicted in FIG. 3. For example, finger grips 120A and 120C on arm 110A may be configured to accommodate a user's index finger and middle finger, respectively, with finger grip 120B on arm 110B being configured to accommodate a user's thumb.

When more than one finger grip 120, such as 120A and 120C are positioned on an arm 110, it may be desirable to offset them from each other. In an exemplary embodiment, a finger grip 120A may be positioned on an outer surface 170 of an arm 110A with a finger grip 120C being positioned on or near an edge of arm 110A between the outer surface 170 and an inner surface 160. It also may be desirable, when forceps 100 includes more than one finger grip 120 on an arm 110, to position a finger grip 120A at a different distance from the hinge 130 than finger grip 120C on arm 110A. Although using two finger grips 120 on a single arm 120 is described above, more than one arm 110 may include multiple finger grips 120 and a particular arm 110 may include more than two finger grips 120.

In some embodiments, the positioning of finger grips 120 on an arm 110 may be adjustable or fixed. Finger grips 120 may be positioned on approximately the middle third of the length of the forceps. Finger grips 120 may be positioned proximate a center of an arm 110 and/or on an exterior surface 170 of an arm. It may be advantageous to position the finger grips 120 on an exterior surface 170 of an arm 110 to increase control of forceps 100. Finger grips 120 may be positioned at approximately the same position on each arm 110 of forceps 100 or at different positions. Finger grips 120 may be positioned on arms 110 of forceps 100 so that the hinge 130 contacts the user's palm during use. Finger grips 120 may be positioned on their respective arms 110 so that the hinge 130 of the forceps 100 does not contact the user's palm during use. Finger grips 120 may also be positioned on an arm 110 at a distance from an end 140 of the arm so that a user may comfortably hold the forceps 100 in the user's hand while maintaining the desired control of the forceps 100.

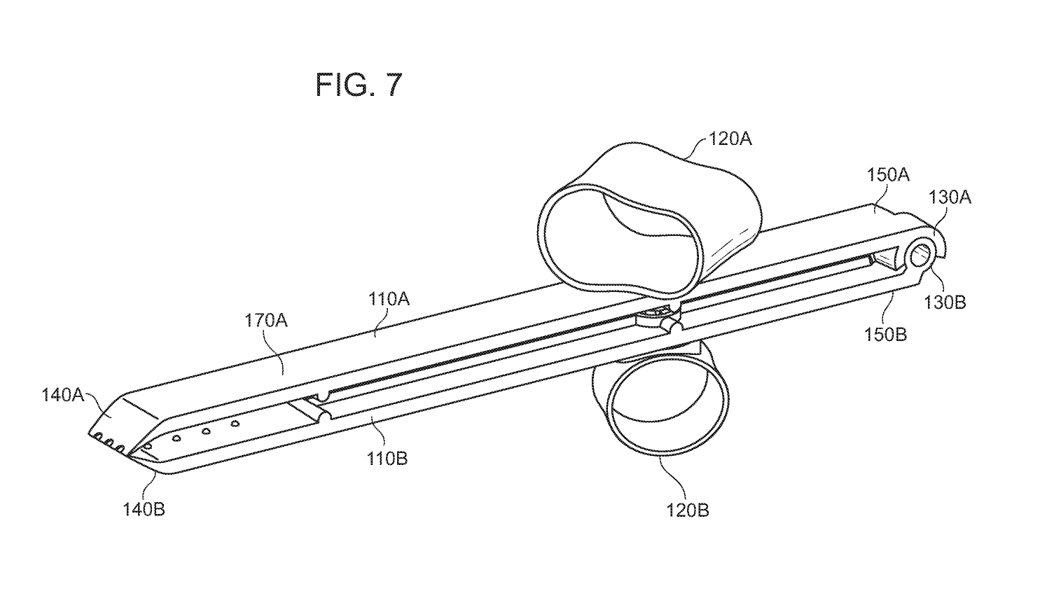

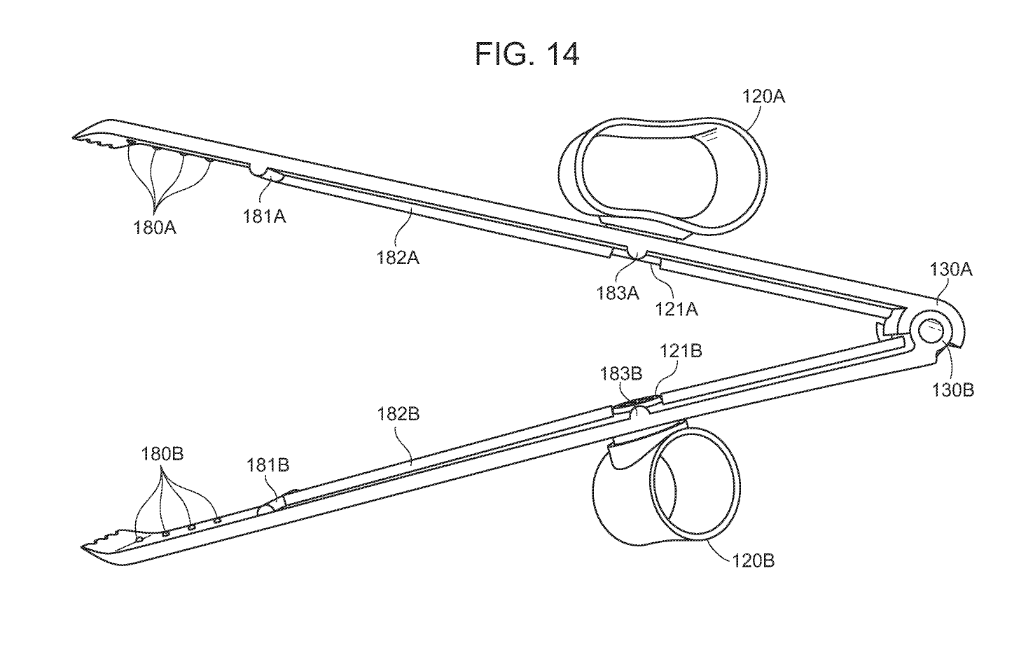

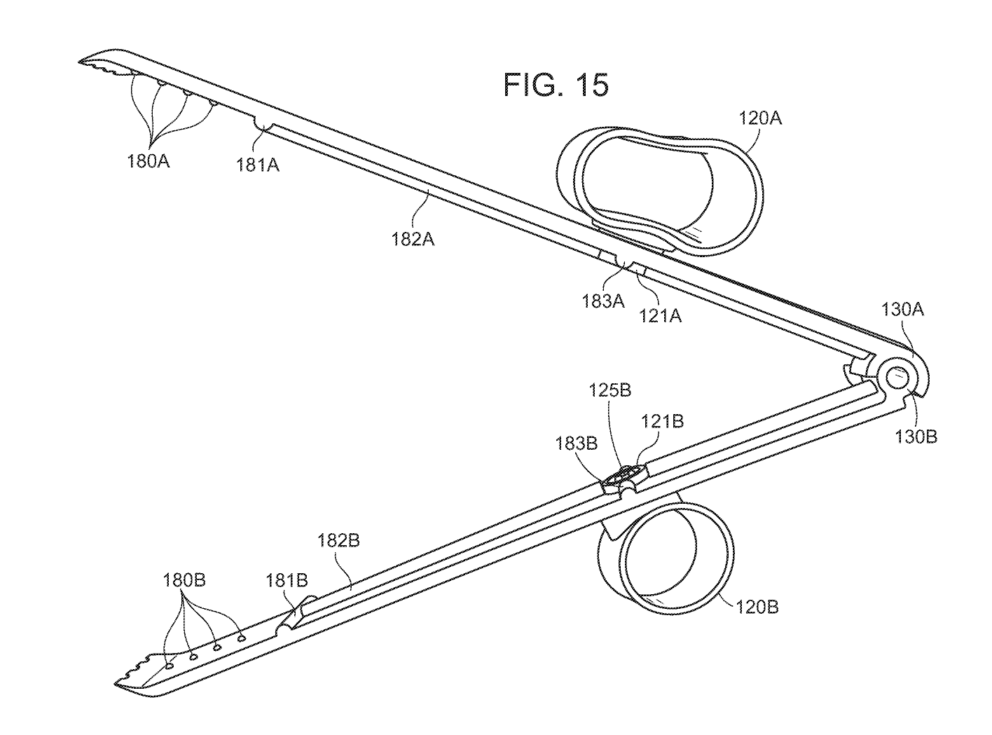

FIGS. 7 to 17 depict an embodiment comprising two finger grips 120A and 120B attached to swivel mounts 121A and 121B, respectively. Swivel mounts 121A and 121B can be any type of mechanism that allows finger grips 120A and 120B to rotate within their respective arms 110A and 110B, which could include a ball and socket, or some type of cylinder within a fitting, for example. In this embodiment, finger grip 120A is configured to accept a plurality of fingers, such as an index finger and a middle finger, and finger grip 120B is configured to accept a single finger, such as thumb. In this embodiment, the position of a user's hand may vary in accordance with the manner in which the user grips the forceps 100. For example, a user's palm is near the hinge 130, the openings of finger grips 120A and 120B may be aligned with the length of arms 110A and 110B.

However, if the user's palm is on a side of forceps 100, the openings on finger grips 120A and 120B may be configured perpendicular to the length of arms 110A and 110B. These configurations are just examples, as a user may choose to swivel finger grips 120A and 120B in different embodiments.

In some embodiments, swivel mounts 121A and 121B may be configured to lock in position to prevent rotation of finger grips 120A and 120B unless a user specifically chooses to unlock the swivels and adjust the rotation. In some embodiments, the swivel mounts 121A, 121B may be configured to rotate between several fixed positions. In other embodiments, swivel mounts 121A, 121B may be configured to allow finger grips 120A and 120B to freely rotate within a defined range. In still other embodiments, swivel mounts 121A, 121B may be configured to allow finger grips 120A and 120B to rotate continuously.

In some embodiments, one or more finger grips 120 may be positioned approximately perpendicular to or oblique to a surface of the arm 110. A finger grip 120 may be positioned obliquely relative to a surface of an arm 110, as depicted in FIGS. 2A and 2B. Positioning a finger grip 120 obliquely to a surface of arm 110 may increase user comfort. For example, during surgery, a user may find it more comfortable to control and/or retain arms 110 of forceps 100 using finger grips 120 rather than by contacting the arms with the user's fingertips.

The size and/or shape of a finger grip 120 may vary. A finger grip 120 may be large enough for one or more of a user's fingers to be placed at least partially in the finger grip 120, such as finger grip 120A as shown in 7. In an embodiment, a finger grip 120 may only be large enough for the user to position one finger at least partially in the finger grip 120, such as finger grip 120B as shown in FIGS. 7 to 17. A finger grip 120 may have a circular, semi-circular, oval, oblong, square, rectangular, or an irregular shape. A finger grip 120 may be in the shape of a ring, c-shaped ring, an oval-shaped ring, or thimble shaped. A finger grip 120 may be designed such that a user may be able to position a finger completely through the finger grip 120. The opening of the finger grip 120 may be sized so that only a portion of a user's finger may pass through an opening.

In an exemplary embodiment, a finger grip 120 may cover at least a portion of an end of a user's finger. A finger grip 120 may comprise a single opening and be configured to allow a user to place a finger within the finger grip 120 as opposed to passing a finger through the finger grip 120. A finger grip 120 may be comprised of a flexible material. For example, the finger grips 120 may approximately or at least partially conform to the shape of a user's finger. In an embodiment, a finger grip 120 may be similar to a tip of a surgical glove. In some embodiments, a finger grip 120 may be comprised of a mesh material or otherwise comprise a plurality of openings that are configured to allow the passage of air but not the passage of a finger.

In an embodiment, a finger grip 120 may be formed from a material similar to or different from the arms 110 and/or hinge 130 of the forceps 100. The finger grips 120 may be formed of a plastic, such as latex, rubber, and/or metal, such as stainless steel. The finger grips 120 may be formed of a material that is configured to be disposed of after one use. The finger grips 120 may be formed of any material capable of being sterilized and/or autoclaved. The finger grips 120 may be formed of a food-safe material. Each finger grip 120 may be comprised of different materials or possess a different configuration from other finger grips 120.

A finger grip 120 may facilitate movement and/or control of an arm 110 of a forceps 100 by a user. It may be desirable to include finger grips 120 on a forceps 100 to reduce strain on a user's hand 200. During use, a user may only need to apply pressure to the forceps 100 to grip an object 210 and not to retain the forceps 100 in the user's hand 200 since the finger grips 120 would inhibit the forceps 100 from being dropped by the user.

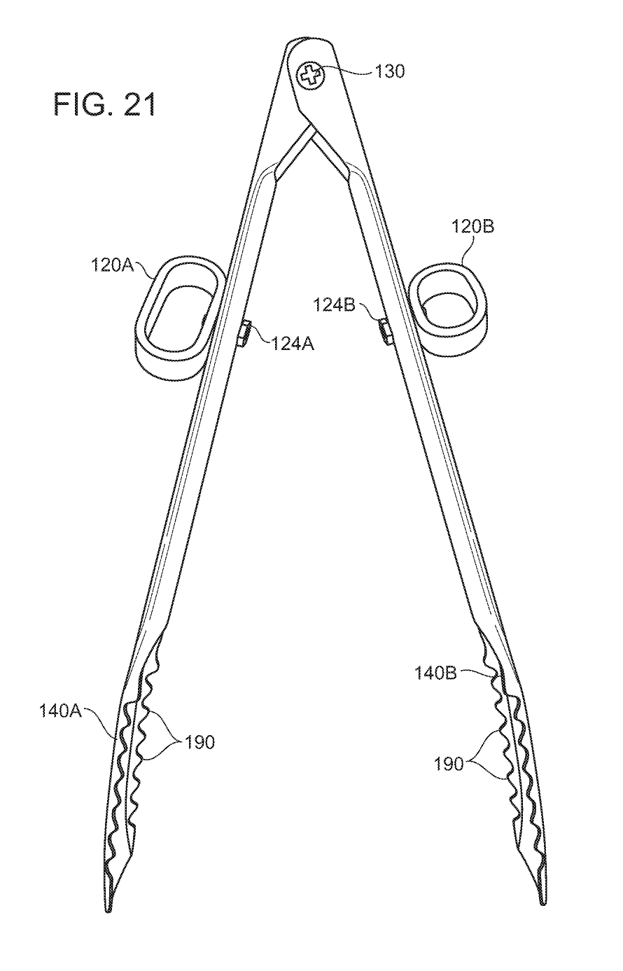

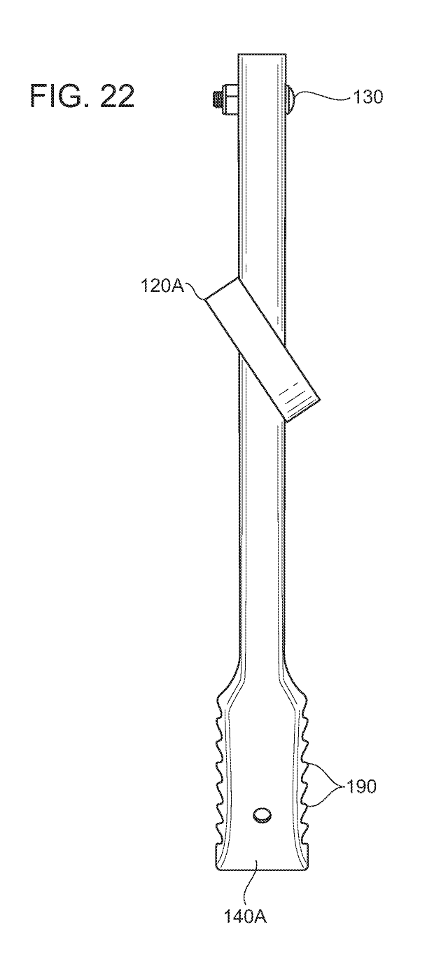

A hinge 130 may couple two or more arms 110 of forceps 100 together, as depicted in FIGS. 1 to 24D. In certain embodiments, the hinge 130 may be positioned at a proximate end 150 of each of the arms 110 of forceps 100. In an embodiment, the hinge 130 may be a pin in an opening at an end 150 of each arm 110. The hinge 130 and/or the configuration of the proximate ends 150 may allow no greater than 30 degrees of separation, at least 30 degrees of separation, at least 45 degrees of separation, at least 60 degrees of separation, at least 75 degrees of separation, at least 90 degrees of separation, at least 135 degrees of separation, or at least 180 degrees of separation between coupled arms 110.

The hinge 130 and/or the configuration of the proximate ends 150 may allow substantially uninhibited movement of at least one of the arms 110 relative to the other arm 110. Utilizing forceps 100 with a hinge 130 capable of such uninhibited movement may be advantageous when compared with forceps 100 with fused or spring loaded hinges. The hinge 130 may include a ball bearing. Fused or spring loaded hinges 130 typically require a substantial force from a user to alter the position of one arm 110 relative to the other arm 110. A user will typically experiences less hand fatigue and/or strain during prolonged or repetitive use of such a forceps 100 since less force is required to move the arm 110 of the forceps 100.

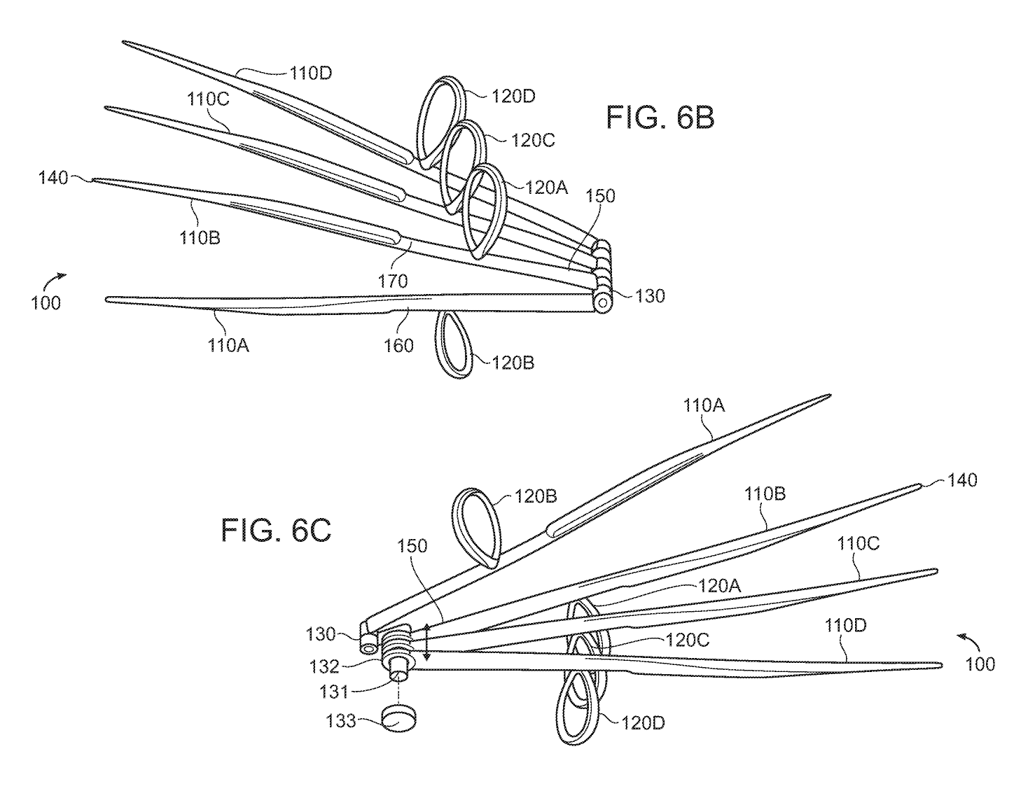

In the embodiment shown in FIG. 6A, the forceps 100 include three arms 110A, 110B, 110C. It should be appreciated that any number of arms 110 greater than two may be utilized in various embodiments and that the present disclosure should not be construed as limited to any particular number of arms 110 illustrated in the exemplary figures. FIG. 6B illustrates an embodiment in which four arms 110A, 110B, 110C, 110D are provided. Although not shown, an embodiment may be provided with five arms 110 to accommodate four fingers and a thumb of the user.

The proximate end 150 of each arm is attached to a pin that serves as hinge 130. In this embodiment, arm 110A and 110C are free to move independently of each other meaning that the angle between arm 110A and 110B is not mechanically affected by the angle between arm 110B and 110C. (However, the limitations of a user's hand may impose practical limitations on the movements of arms 110A and 110C). In some embodiments, arms 110A and 110C may be configured such that only one of them may be in contact with arm 110B at a given instance. In other embodiments, arm 110B is configured to contact either arm 110A, arm 110C, or both without limitation.

In some embodiments, the arms 110 may be adjusted to spread or contract with respect to each other. In such embodiments, one or more of the arms 110 may be adapted to be moved (such as by sliding) along a pin 131. Such one or more of the arms 110 may be adapted to be releasably secured in various positions along the pin 131. By adjusting one or more of the arms 110, the effective spread or width between the arms 110 may be adjusted for different applications.

FIG. 6C illustrates an embodiment in which the arms 110 may be adjusted between various positions with respect to each other so as to effectively adjust the width or spread between the arms 110. In the embodiment shown in FIG. 6C, four arms 110A, 110B, 110C, 110D are illustrated. The first arm 110A is shown as being connected to a hinge 130. A pin 131 extends from the hinge 130. In the exemplary embodiment shown in FIG. 6C, the pin 131 includes a curve or angled portion; with a first portion of the pin 131 extending at a right angle with respect to a second portion of the pin 131.

Continuing to reference FIG. 6C, it can be seen that three arms 110B, 110C, 110D are pivotably or rotatably connected to the second portion of the pin 131. In such an exemplary embodiment, each of the arms 110B, 110C, 110D may include an arm connector 132 which is pivotably connected around the pin 131. Although various configurations may be utilized, the exemplary embodiment in the figures illustrates that the arm connectors 132 each comprise rings which extend around the pin 131 in a rotatable manner. A cap 133, washer, or other securing mechanism may be utilized to removably secure the arms 110B, 110C, 110D at a specific distance (spread) with respect to each other. The arms 110B, 110C, 110D may be pushed together to contract and then secured with the cap 133, or may be spread apart and then secured with the cap 133. Thus, the effective spread of the arms 110 may be adjusted using the pin 131 and arm connectors 132, locked in place with the cap 133.

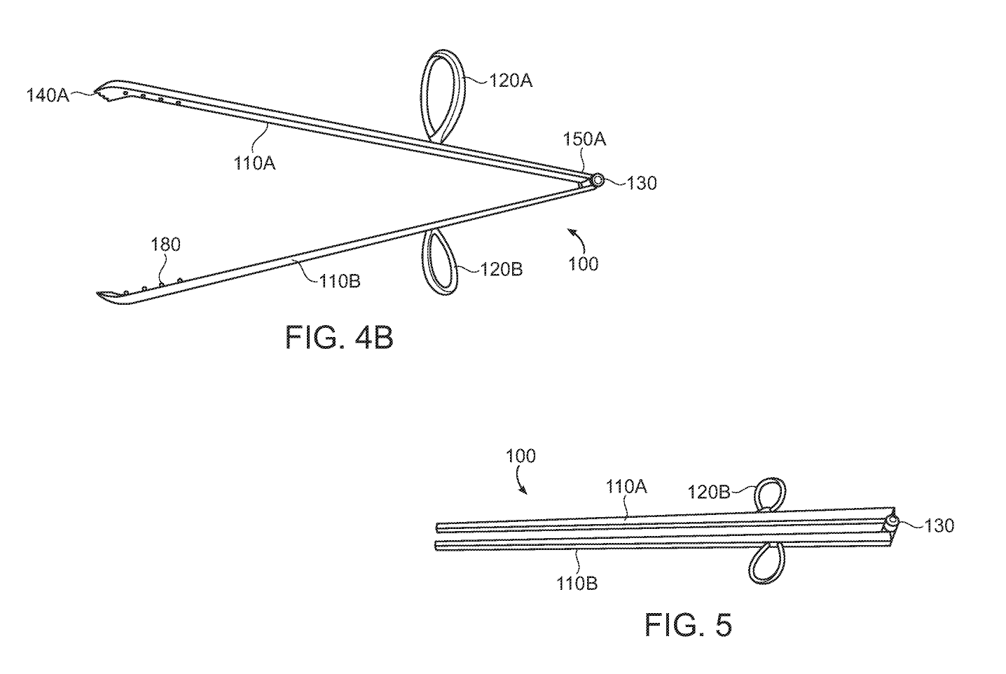

In some embodiments, the forceps 100 may be designed for specific uses, such as medical, food service, eating, cooking, semiconductor fabrication, or small-scale laboratory experiments. FIGS. 4A and 4B depict embodiments of forceps 100 designed for food service applications. Forceps 100 may include tong-like arms 110. Finger grips 120 may be positioned on arms 110. Such finger grips 120 may be positioned on arm 110 at a distance from an end 140 of the arm so that a user may comfortably hold the forceps in the user's hand.

Finger grips 120 may be conduits and/or made from a flexible material, see FIG. 4A. In an exemplary embodiment, finger grips 120 may be circular and resist deformation, see FIG. 4B. An arm 110 of forceps 100 may be serrated at end 140, as depicted in FIGS. 4A and 4B. End 140 of arm 110 of forceps 100 may include protrusions 180. Protrusions 180 may facilitate retaining food in ends 140 of forceps 100. Hinge 130 of forceps 100 may allow arms 110 to be separated by no greater than 30 degrees of separation, at least 30 degrees of separation, at least 45 degrees, at least 60 degrees, at least 75 degrees, at least 90 degrees, at least 135 degrees, or at least 180 degrees.

The hinge 130 may function to allow a predetermined separation between the arms 110 of the forceps 100. Allowing a large separation of the arms 110 may allow a user to grab large objects 210 with the forceps 100. For example, a user may be able to grab large chicken breasts, ribs, and other food that may not be easily grasped with currently available commercial tongs. In addition, using forceps 100 with a hinge 130 allows a user to grab small and large objects 210 with the same forceps 100. For example, a restaurant may use a single forceps 100 that allows enough separation between arms 110 of the forceps 100 for a user to grab a large chicken breast and a small enough separation between arms 110 for a user to grab a small chicken wing with the forceps.

FIGS. 7 to 17 show various view of an alternate embodiment of hinged forceps 100. This embodiment is preferentially comprised of plastic, but is not limited to any particular type of material. In this embodiment, finger grip 120A is configured to simultaneously accept an index finger and a middle finger, and finger grip 120B is configured to accept a thumb from that same hand. However, finger grip 120A can be used with a single finger—not necessarily the index finger or middle finger. Similarly, if finger grip 120A is being used with a thumb, finger grip 120B could be used with some other finger on that hand.

In this embodiment, forceps 100 comprises protrusions 180 that are small bumps that assist with the gripping of objects as well as protrusions 181 which run along with the width of arms 110A and 110B. In addition to assisting with the gripping of objects, protrusions 181 assist with keeping gripped objects near the distal ends 140A and 140B of arms 110A and 110B, respectively. Other embodiments may include different numbers and configurations of protrusions 181.

This embodiment is substantially comprised of four parts that can each be constructed of a single piece of material. In this embodiment, the four parts include an upper arm 110A, a lower arm 110B, and finger grips 120A and 120B. It should be appreciated that the designation of 110A as upper arm and 120B as lower arm is for sake of convenience as forceps 100 are not limited to operating in any particular orientation.

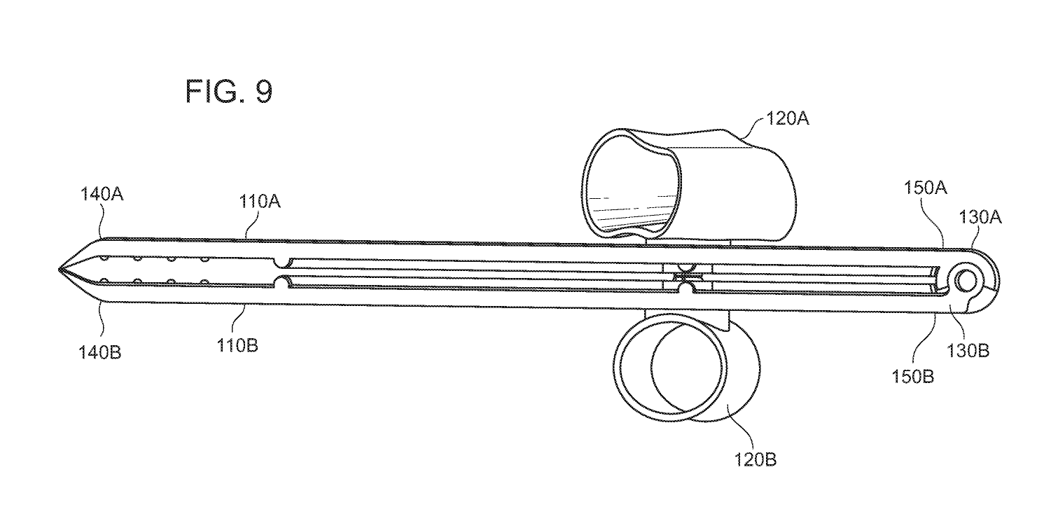

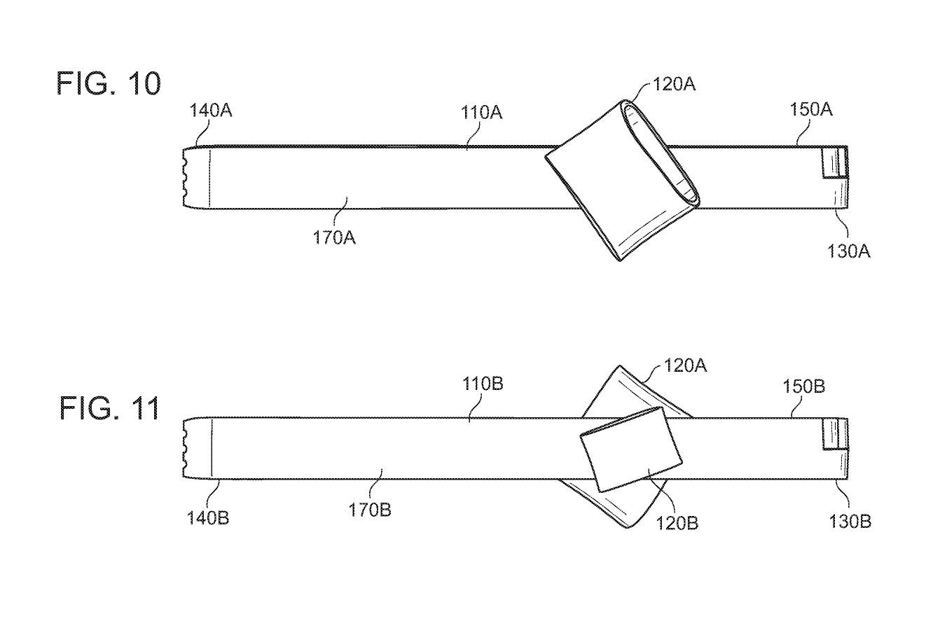

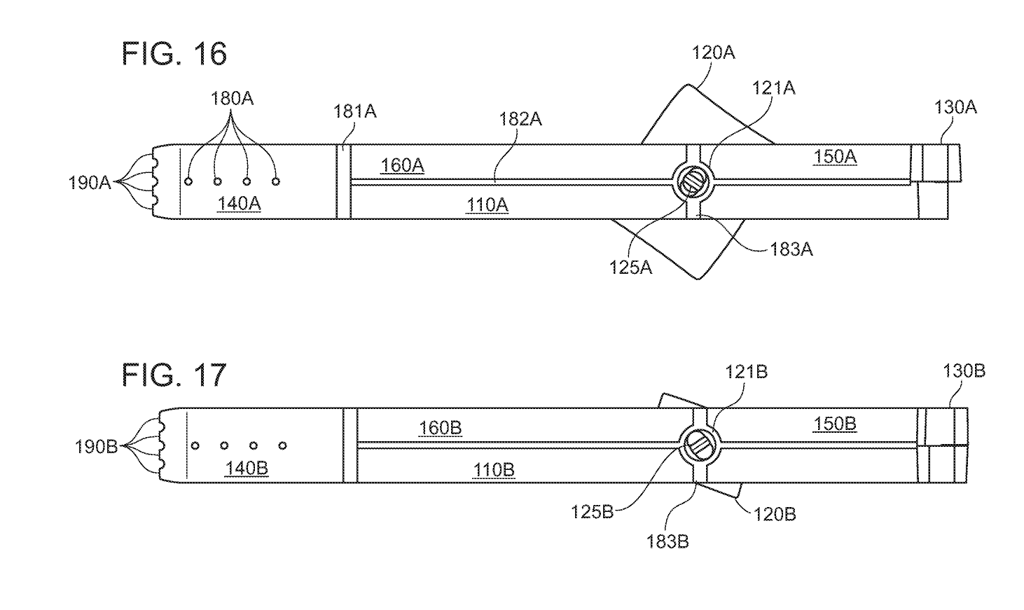

FIGS. 10 and 16 show the exterior surface 170A and the interior surface 160A of upper arm 110A. FIGS. 11 and 17 show the exterior surface 170B and the interior surface 160B of lower arm 110B. In this embodiment, hinge 130 is comprised of two portions 130A and 130B that are part of arms 110A and 110B, respectively, as best illustrated in FIGS. 9 and 13. In this embodiment, upper arm 110A contains a hinge portion 130A comprising a socket that is configured to accept a bar within hinge portion 130B of lower arm 110B. When pressed together with sufficient force, the two hinge portions 130A, 130B may snap into place in a manner that allows arms 110A, 110B to swing with respect to each other. As shown in FIG. 13, hinge portions 130A and 130B can be comprised of both a socket and bar configured in a manner that allows them to snap into place. This type of hinge configuration is simply an example. Other hinge configurations are suitable for use with hinged forceps 100.

This embodiment also includes finger grips 120A and 120B that configured to couple with arms 110A, 110B. In this embodiment, finger grip 120A is adapted to simultaneously receive two fingers and finger grip 120B is adapted to receive a single finger. However, finger grips 120A, 120B could have the same shape or different shapes. As shown in FIGS. 16 and 17, arms 110A and 110B comprise swivel mounts 121A, 121B, respectively. In this embodiment, swivel mounts 121A, 121B comprise circular openings configured to receive swivel posts 125A, 125B that are coupled to finger grips 120A, 120B. In this embodiment, the swivel posts 125A, 125B and swivel mounts 121A, 121B are configured to form a snap-fit joint. In particular, swivel posts 125A, 125B are hollow cylinders with cutouts and ridges on the end.

The diameter of the swivel posts 125A, 125B may be less than the diameter of the swivel mounts 121A, 121B except at the ridges where they are generally greater than the diameter of the swivel mounts 121A, 121B. During assembly, a swivel post 125 may be inserted into a swivel mount 121. In this embodiment, because the swivel post 125 is hollow with cut-outs, it will compress when inserted into the swivel mount 121 sufficiently for the ridge to pass through its opening. Once the ridge has passed through swivel mount 121, the swivel post 125 will return to its normal shape. Because swivel mount 121 of this embodiment is circular, the swivel post 125 of the corresponding finger grip 120 can freely rotate. In some embodiments, the range of rotation can be limited. In still other embodiments, swivel posts 125 and swivel mounts 121 are configured to allow a finite number of fixed rotations.

The embodiment may also include supports and protrusions that can optionally be used with a hinged forceps 100. In this embodiment, the arms 110 each have an arm support 182 that runs partially along the length of each arm 110. The use of an arm 182 can provide structural support to help maintain the shape of its arm 110. In addition, this embodiment includes a bar protrusion 181 and a finger grip support 183. Bar protrusion 181 can be used to help grip objects as well as provide structural support for its arm 110 near the location where gripping occurs. Similarly, finger grip support 183 provides additional to its arm 110 near the location of its finger grip 120. The number and configuration of additional support structures, if any, can be designed based on the expected usage of the hinged forceps 100.

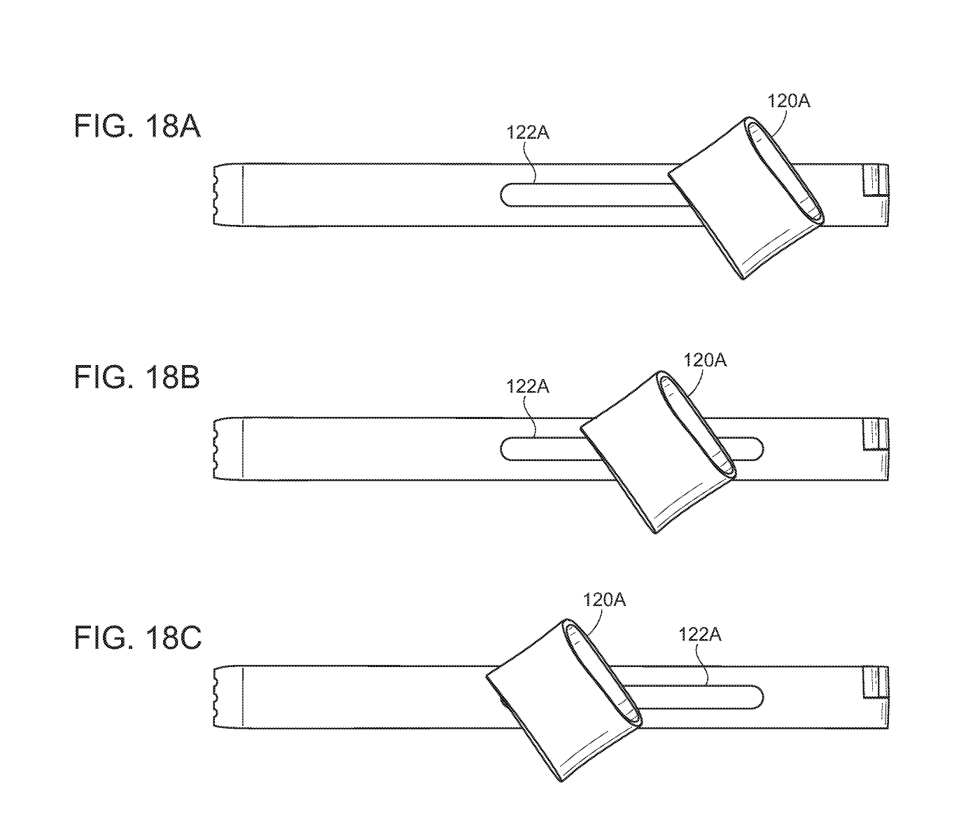

FIGS. 18A, 18B, and 18C illustrate an embodiment comprising a slot 122A which is configured to receive a finger grip 120A. In this embodiment, finger grip 120A is configured to slide along the length of the slot 122A. FIG. 18A illustrates the finger grip at a first location. FIG. 18B illustrates the finger grip 120A at a second location. FIG. 18C illustrates the finger grip 120A at a third location. In some embodiments, slot 122A comprises ridges that limit the position of finger grip 120A to a few specific locations. In other embodiments, finger grip 120A is free to move long the length of slot 122A. In addition, the friction between finger grip 120A and slot 122A can be altered to vary the ease at which the finger grip can move along the slot 122A. In some embodiments, finger grip 120A can be locked into a position that can only be altered by first unlocking it.

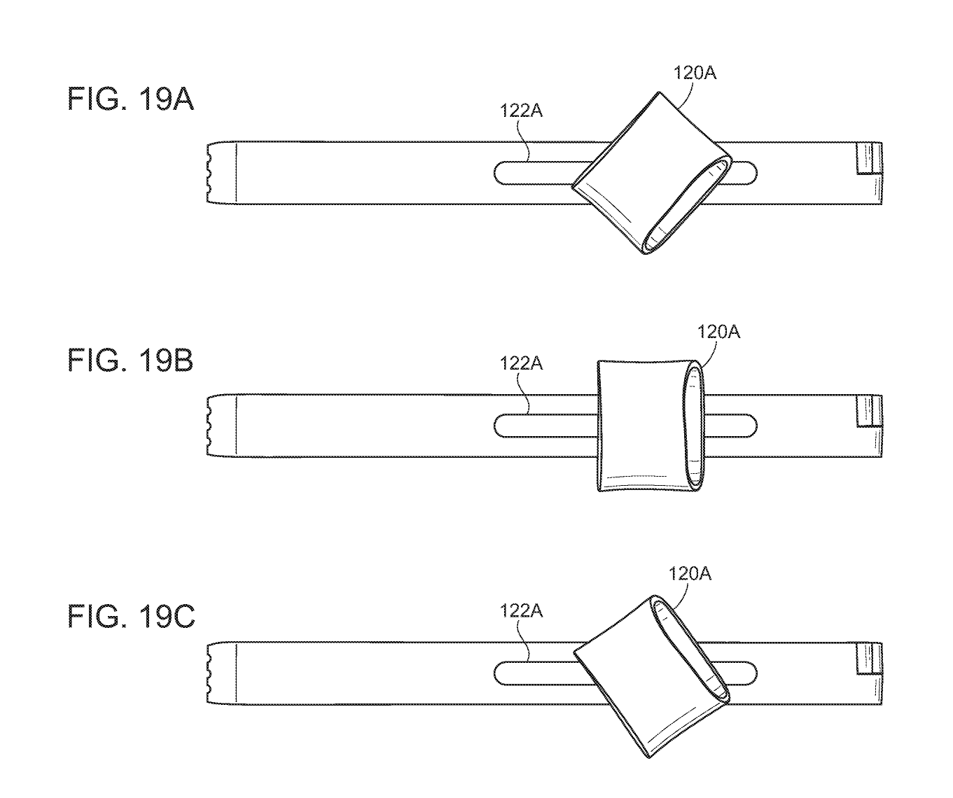

FIGS. 19A, 19B, and 19C illustrate rotating the orientation of finger grip 120A within swivel mount 121A (not shown). FIG. 19A illustrates the finger grip at a first orientation. FIG. 19B illustrates the finger grip 120A at a second orientation. FIG. 19C illustrates the finger grip 120A at a third orientation. In some embodiments, slot 122A comprises ridges that limit the position of finger grip 120A to a few specific locations. In other embodiments, finger grip 120A is free to move long the length of slot 122A. In addition, the friction between finger grip 120A and slot 122A can be altered to vary the ease at which the finger grip can move along the slot 122A. In some embodiments, finger grip 120A may be locked into a position that can only be altered by first unlocking it. Some embodiments may include a swivel mount 121 and a slot 122 enabling a finger grip 120 to be rotated and laterally positioned relative to its arm 110.

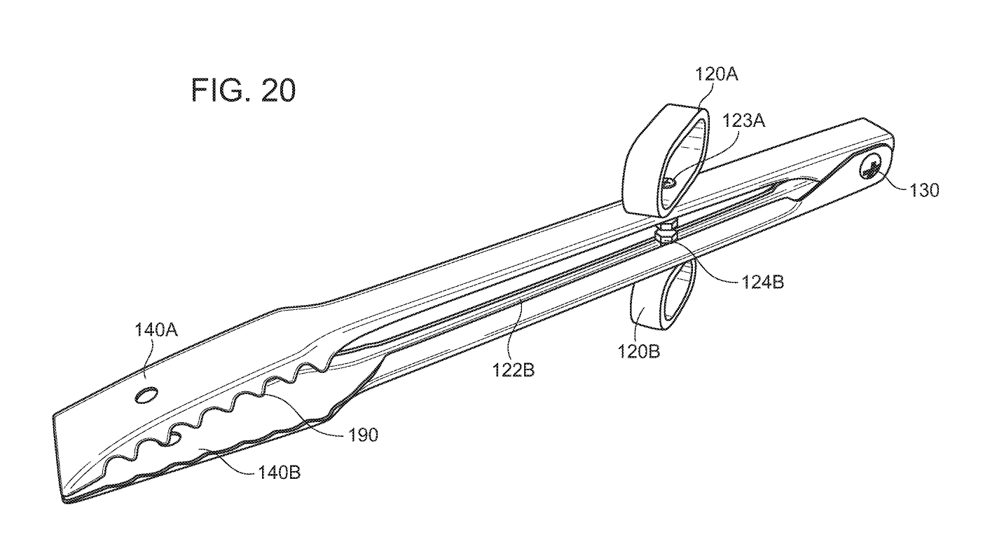

In the embodiment shown in FIGS. 20-22, forceps 100 may comprise two arms 110A and 110B, a hinge 130, two distal ends 140A and 140B, with each distal end comprising a plurality of teeth 190. Such an exemplary embodiment is preferentially comprised of metal, but is not limited to a particular type of material. In this embodiment, the teeth 190 on distal ends 140A and 140B may be configured to interlock such that the teeth 190 on distal end 140A are configured to touch the gaps between teeth 190 on distal end 140B and vice versa. This embodiment may also allow the finger grip to rotate. In such an embodiment, finger grip 120 may comprise a bolt 123 that passes through an opening in its arm 110. By tightening a nut 124 on this bolt 123, finger grip 120 can be held in place. This nut 124 can be loosened to permit finger grip 120 to rotate. If the opening in arm 110 comprises a slot 122, the finger grip 120 will be able to rotate as well as move along the slot 122. This nut 124 can be tightened again to fix finger grip 120 in its new location. Some embodiments include a number of fixed lateral positions for finger grip 120. Other embodiments may permit repositioning at arbitrary locations within a defined region along arm 110, such as a slot 122 or the entire length of an arm 110.

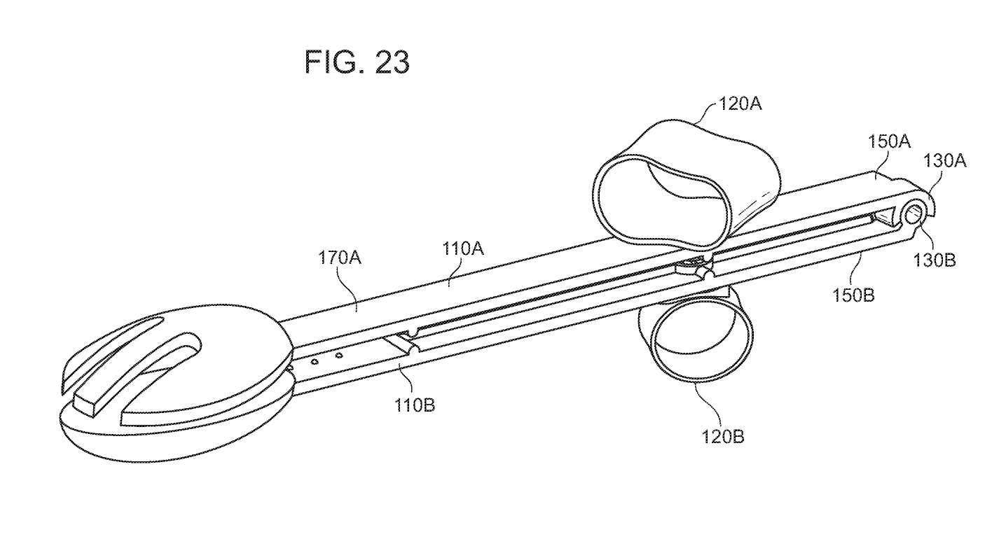

In another embodiment, forceps 100 may be an eating and/or cooking utensil, such as a chopstick, as depicted in FIG. 23. The forceps 100 may be formed from a food-safe material. Arms 110 of the forceps 100 may have a substantially square cross-section. Arms 110 of forceps 100 may taper. At least one finger grip 120 may be positioned on each arm 110. Finger grip 120 may be positioned at a distance from hinge 130 such that a user may comfortably hold the chopstick-like forceps. It may be easier for a user to eat and/or cook with forceps 100 with finger grips 120 than currently available chopsticks since the forceps 100 are easier to operate with the finger grips.

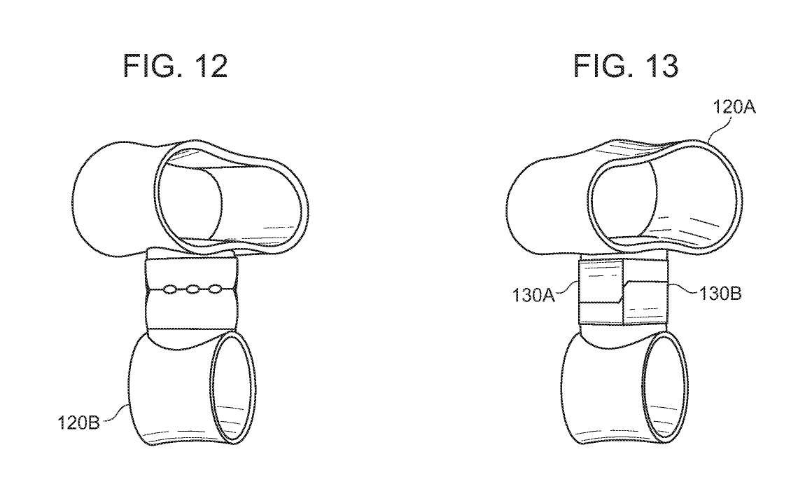

In the embodiment shown in FIG. 12, distal end 140A of arm 110A is configured like a spoon, and distal end 140B of arm 110B is configured like a spork. Other utensil-like configurations are contemplated by the disclosed hinged forceps.

### C. Operation of Preferred Embodiment

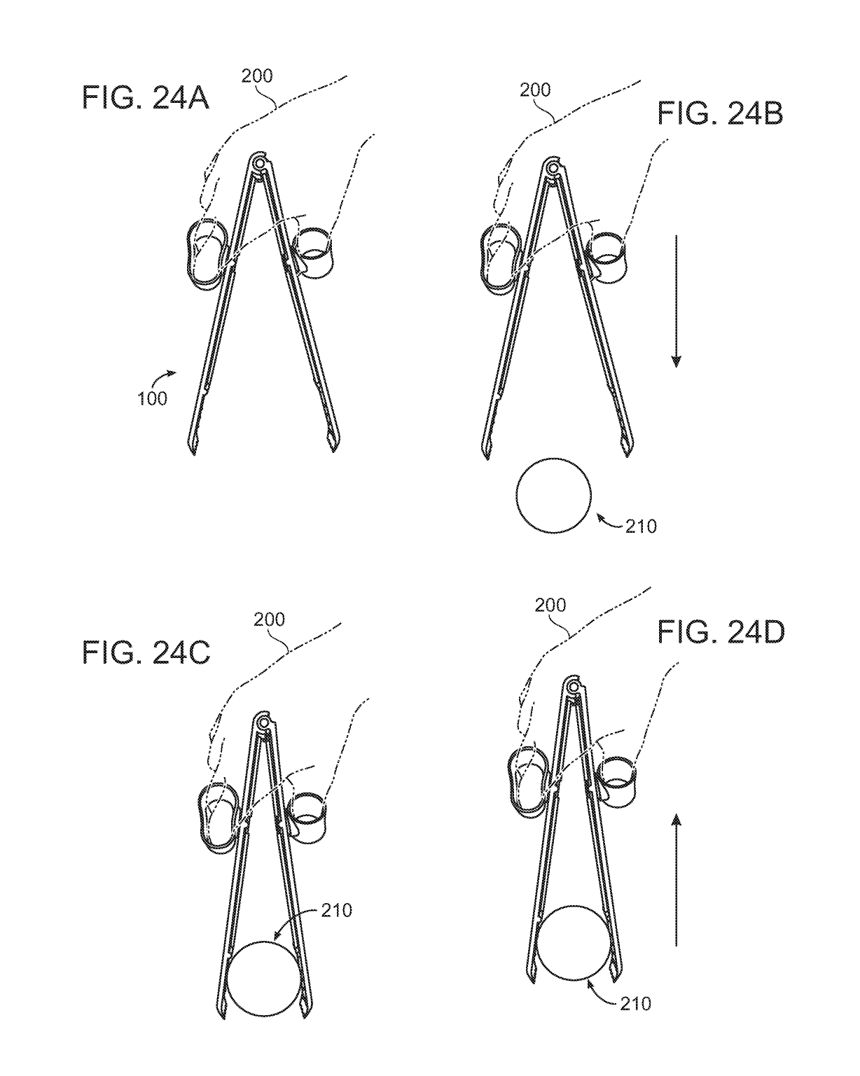

The embodiments described herein can be used for various tasks. As shown in FIGS. 24A-D, hinged forceps 100 can be used to grip and position an object 210. FIG. 24A illustrates a hand 200 positioned within hinged forceps 100 in preparation for use. FIG. 24B illustrates the hinged forceps being lowered over an object 210. In the illustration of FIG. 24B, the arms have been separated sufficiently to allow them to enclose object 210. Once the hinged forceps have been lowered around object 210, hand 200 pushes the arms together to contact the surface of object 210. Because hinged forceps 100 can be used for gripping objects with varying characteristics, such as hardness and texture, bump protrusions 180 and teeth 190 help prevent the object from shifting within the forceps 100. When positioned as shown in FIG. 24C, a user can move the object to any desired location(s) while maintaining a secure grip on the object 200. Once gripping of the object is no longer desired the arms of hinged forceps 100 are separated and lifted away from object 210, which will return the hinged forceps to the status shown in FIG. 24A. Although shown gripping a singe spherical object 210, hinged forceps 100 are not limited to particular shape, configuration, or quantity of objects. Moreover, depending on the characteristics of the object, gripping may involve varying involvement of bump protrusions 180, bar protrusion 190, and/or teeth 190.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

What is claimed is:

1. A forceps comprising:
a plurality of elongated arms, each of the elongated arms comprising a first end and a second end;
a hinge coupling the elongated arms together at the first end of each elongated arm such that the forceps can secure an object between the elongated arms;
at least one finger grip configured to receive at least one finger, wherein the finger grip is attached to an exterior surface of each of the elongated arms between the first end and the second end of the elongated arm to which the finger grip is attached;
wherein the second end of at least one of the elongated arms is configured to aid in securing the object between the elongated arms; and
wherein at least one finger grip is configured to swivel along an axis that is substantially perpendicular to the axis between the first end and the second end of the arm to which said finger grip is attached.

2. The forceps of claim 1, wherein the at least one finger grip is configured to receive at least two fingers.

3. The forceps of claim 1, wherein the second end of at least one of the elongated arms comprises teeth.

4. The forceps of claim 1, wherein at least one finger grip is adjustably attached such that its position on the exterior surface of the elongated arm to which it is attached can be altered.

5. The forceps of claim 4, wherein the at least one adjustably attached finger grip is attached using a slot.

6. The forceps of claim 1, wherein the at least one finger grip is attached using at least one bolt.

7. The forceps of claim 1, wherein the at least one finger grip is attached using a ball and socket.

8. The forceps of claim 1, wherein the at least one finger grip is attached using a snap fit joint.

9. The forceps of claim 1, wherein at least one second end of at least one of the elongated arms is shaped like an eating utensil.

10. The forceps of claim 1, comprising at least three elongated arms.

11. The forceps of claim 10, wherein at least two of the elongated arms are on one side of the forceps and are configured to move independently of each other.

12. The forceps of claim 1, wherein at least one of the elongated arms includes at least two finger grips attached to it.

13. The forceps of claim 1, wherein at least one finger grip has a cylindrical shape with at least two openings configured to allow a finger to enter the finger grip through a first opening and to exit the finger grip through a second opening.

14. The forceps of claim 1, wherein at least one finger grip has a cylindrical shape with no more than one opening configured to receive a finger.

15. A forceps comprising:
a plurality of elongated arms comprising a first end and a second end;
a hinge coupling the elongated arms together at the first end of each arm such that the forceps can secure an object between the elongated arms;
at least one finger grip configured to receive at least one finger, the at least one finger grip being attached to an exterior surface of each of the elongated arms between the first end and the second end of the elongated arm to which said finger grip is attached.
wherein the second end of at least one of the elongated arms is configured to aid in securing the object between the elongated arms; and
wherein at least one finger grip is adjustably attached to one of the elongated arms such that its position on the exterior surface of the elongated arm to which it is attached can be altered.

16. The forceps of claim 15, wherein at least one finger grip is configured to swivel along an axis that is substantially perpendicular to the axis between the first end and the second end of the arm to which said finger grip is attached.

17. The forceps of claim 15, wherein at least one of the elongated arms is shaped like a chop stick.

18. A forceps comprising:
a plurality of elongated arms comprising a first end and a second end;
a hinge coupling the elongated arms together at the first end of each elongated arm such that the forceps can secure an object between the elongated arms;
a first finger grip configured to receive at least one finger, wherein the first finger grip is attached to an exterior surface of a first elongated arm between the first end and the second end of the first elongated arm;
a second finger grip configured to receive at least two fingers, wherein the second finger grip is attached to an exterior surface of a second elongated arm between the first end and the second end of the second elongated arm;
wherein at least one second end of at least one of the elongated arms comprises teeth and is configured to aid securing the object between the elongated arms;
wherein at least one finger grip is adjustably attached to its elongated arm such that its position on the exterior surface of the elongated arm to which it is attached can be altered; and
wherein at least one finger grip is configured to swivel along an axis that is substantially perpendicular to the axis between the first end and the second end of the elongated arm to which said finger grip is attached.

19. The forceps of claim 18, wherein the hinge is integrated with at least one of the elongated arms.

20. The forceps of claim 18, wherein at least one finger grip is positioned obliquely relative to a surface of the arm.
